SEQUENCE LISTING (7 sequences)

SEQ ID NO: 1 - protein, 9 aa, synthetic construct
LHDFRHQIL

SEQ ID NO: 2 - protein, 9 aa, synthetic construct
LYEIKHQIL

SEQ ID NO: 3 - protein, 9 aa, synthetic construct
LYDIRHQIL

SEQ ID NO: 4 - protein, 14 aa, synthetic construct
  VARIANT 1: note = X may be present or absent and when present is E
  VARIANT 2: note = X may be present or absent and when present is L
  VARIANT 3: note = X may be present or absent and when present is Q
  VARIANT 4: note = X may be present or absent and when present is A or T
  VARIANT 5: note = X is T or E
  VARIANT 7: note = X is H or Y
  VARIANT 8: note = X is D or E
  VARIANT 9: note = X is F or I
  VARIANT 10: note = X is R or K
XXXXXLXXXXHQIL

SEQ ID NO: 5 - protein, 14 aa, synthetic construct
ELQTELYEIKHQIL

SEQ ID NO: 6 - protein, 12 aa, synthetic construct
QTELYEIKHQIL

SEQ ID NO: 7 - protein, 10 aa, synthetic construct
ELYEIKHQIL

(19) United States
(12) Patent Application Publication
(10) Pub. No.: US 2024/0390452 A1
(43) Pub. Date: Nov. 28, 2024

(54) Protective Agent Against Endothelial Dysfunction

(71) Applicant: THE TRUSTEES OF THE UNIVERSITY OF PENNSYLVANIA, Philadelphia, PA (US)

(72) Inventors: Shampa Chatterjee, Philadelphia, PA (US); Aron Fisher, Philadelphia, PA (US); Oindrila Paul, Philadelphia, PA (US)

(21) Appl. No.: 18/674,214

(22) Filed: May 24, 2024

Related U.S. Application Data
(60) Provisional application No. 63/469,255, filed on May 26, 2023.

(51) Int. Cl. A61K 38/08; A61K 9/127; A61P 39/06

(52) U.S. Cl. CPC A61K 38/08; A61K 9/127; A61P 39/06

(57) ABSTRACT

Described herein is a method of treating, ameliorating, and/or preventing endothelial dysfunction caused by SARS-CoV-2 in a subject in need thereof. Also described herein is a method of treating and/or ameliorating a coronavirus infection. Both methods include administering to the subject an effective amount of a peptide capable of inhibiting the phospholipase A2 (PLA2) activity of peroxiredoxin 6 (Prdx6).

### CROSS-REFERENCE TO RELATED APPLICATIONS

The present application claims priority under 35 U.S.C. § 119 (e) to U.S. Provisional Patent Application No. 63/469,255, filed May 26, 2023, which is incorporated herein by reference in its entirety.

### SEQUENCE LISTING

The XML text file named “046483_7365US1_SequenceListing.xml” created on May 23, 2024, comprising 10,651 bytes, is hereby incorporated by reference in its entirety.

### BACKGROUND

Coronavirus infections, such as SARS-CoV-2 infections, sometimes cause endothelial activation and dysfunction in patients, which are associated with more severe cases of diseases. Therefore, there is a need to treat and/or ameliorate coronavirus infections, such as by treat and/or prevent the endothelial dysfunction. The present invention addresses this need.

### SUMMARY

In some aspects, the present invention is directed to a method of treating, ameliorating, and/or preventing endothelial dysfunction caused by severe acute respiratory syndrome coronavirus 2 (SARS-CoV-2) in a subject in need thereof.

In some embodiments, the method comprising administering to the subject an effective amount of a polypeptide consisting of SEQ ID NO: 4

X¹X²X³X⁴X⁵LX⁶X⁷X⁸X⁹HQIL;

wherein:
   X¹ may be present or absent and when present is E;
   X² may be present or absent and when present is L;
   X³ may be present or absent and when present is Q;
   X⁴ may be present or absent and when present is A or T;
   X⁵ is T or E;
   X⁶ is H or Y;
   X⁷ is D or E;
   X⁸ is F or I;
   X⁹ is R or K.

In some embodiments, the polypeptide is selected from the group consisting of:

(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

In some embodiments, the polypeptide is formulated in a liposome.

In some embodiments, the polypeptide is formulated in a pharmaceutical composition comprising a pharmaceutically acceptable carrier.

In some embodiments, administering the polypeptide to the subject comprises aerosol inhalation, intratracheal injection, intravenous injection, or any combinations thereof.

In some embodiments, the endothelial dysfunction comprises endothelial activation and/or endothelial inflammatory phenotype.

In some embodiments, the endothelial dysfunction occurs at least in pulmonary endothelial tissue of the subject.

In some embodiments, the administration results in prevention of reactive oxygen species (ROS) production from cells of the pulmonary endothelial tissue, or reduction of ROS production compared to a reference level of ROS production prior to administering the polypeptide.

In some embodiments, the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

In some embodiments, the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide.

In some embodiments, the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide

In some embodiments, the administration results in prevention of NLR family pyrin domain containing (NLRP3) expression from cells of the pulmonary endothelial tissue, or reduction of NLRP3 expression compared to a reference level of NLRP3 expression prior to administering the polypeptide.

In some embodiments, the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the administration results in prevention of caspase 1 level from cells of the pulmonary endothelial tissue, or reduction of caspase 1 level compared to a reference level of caspase 1 expression prior to administering the polypeptide.

In some embodiments, the reduction of caspase 1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 level prior to administering the polypeptide. In some embodiments, the subject is a mammal.

In some embodiments, the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

In some embodiments, the subject is a human.

In some embodiments, the subject has active SARS-CoV-2 infection, coronavirus disease 2019 (COVID-19), and/or post-acute sequelae SARS-CoV-2 infection (PASC).

In some embodiments, the SARS-CoV-2 is a SARS-CoV-2 variant.

In some embodiments, the SARS-CoV-2 variant is selected from alpha (B.1.1.7 and Q lineages), beta (B.1.351 and descendent lineages), gamma (P.1 and descendent lineages), delta (B.1.617.2 and AY lineages), epsilon (B.1.427 and B.1.429), zeta (P.2), eta (B.1.525), iota (B.1.526), kappa (B.1.617.1), 1.617.3, mu (B.1.621 and B.1.621.1), and omicron (B.1.1.529 and BA lineages (BA.1, BA.1.1, and BA.2)).

In some aspects, the present invention is directed to a method of treating or ameliorating a coronavirus infection in a subject in need thereof.

In some embodiments, the method comprising administering to the subject an effective amount of a polypeptide consisting of SEQ ID NO: 4

X¹X²X³X⁴X⁵LX⁶X⁷X⁸X⁹HQIL;

wherein:
   X¹ may be present or absent and when present is E;
   X² may be present or absent and when present is L;
   X³ may be present or absent and when present is Q;
   X⁴ may be present or absent and when present is A or T;
   X⁵ is T or E;
   X⁶ is H or Y;
   X⁷ is D or E;
   X⁸ is F or I;
   X⁹ is R or K.

In some embodiments, the polypeptide is selected from the group consisting of:

(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

In some embodiments, the polypeptide is formulated in a liposome.

In some embodiments, the polypeptide is formulated in a pharmaceutical composition comprising a pharmaceutically acceptable carrier.

In some embodiments, administering the polypeptide to the subject comprises aerosol inhalation, intratracheal injection, intravenous injection, or any combinations thereof.

In some embodiments, the coronavirus infection causes endothelial dysfunction in the subject.

In some embodiments, the endothelial dysfunction comprises endothelial activation and/or endothelial inflammatory phenotype.

In some embodiments, the endothelial dysfunction occurs at least in pulmonary endothelial tissue of the subject.

In some embodiments, the administration results in prevention of reactive oxygen species (ROS) production from cells of the pulmonary endothelial tissue, or reduction of ROS production compared to a reference level of ROS production prior to administering the polypeptide.

In some embodiments, the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

In some embodiments, the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide.

In some embodiments, the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide

In some embodiments, the administration results in prevention of NLR family pyrin domain containing (NLRP3) expression from cells of the pulmonary endothelial tissue, or reduction of NLRP3 expression compared to a reference level of NLRP3 expression prior to administering the polypeptide.

In some embodiments, the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the administration results in prevention of caspase 1 level from cells of the pulmonary endothelial tissue, or reduction of caspase 1 level compared to a reference level of caspase 1 expression prior to administering the polypeptide.

In some embodiments, the reduction of caspase 1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 level prior to administering the polypeptide. In some embodiments, the subject is a mammal.

In some embodiments, the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

In some embodiments, the subject is a human.

In some embodiments, the coronavirus comprises at least one selected from the group consisting of SARS-CoV, MERS-CoV, SARS-CoV2, HCoV-OC43, HCoV-HKU1, HCOV-229E, and HCoV-NL63.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1: illustrates certain aspects of the experimental design, in accordance with some embodiments. The experiment measures reactive oxygen species (ROS) production and intercellular adhesion molecule (ICAM-1) expression in human pulmonary microvascular endothelial cell after exposure to serum from either COVID-19 subjects or healthy subjects.

FIGS. 2A-B demonstrate that ROS production decreased post PIP-2 treatment, in accordance with some embodiments. Cells were pre-treated for 3 hrs with PIP-2 and then with COVID-19 serum for an hour. Serum from 5 subjects were used. Cells were then stained with CellROX and imaged for fluorescence. FIG. 2A: Images of the CellROX staining. FIG. 2B: Quantification of the fluorescent signal using Metamorph imaging software *p<0.05.

FIGS. 3A-B demonstrate that ICAM-1 decreased post PIP-2 treatment. Cells were pre-treated for 3 hrs with PIP-2 and then with COVID-19 serum for an hour. Cells were then fixed and ICAM-1 were monitored by immunostaining with anti-ICAM antibody. FIG. 3A: ICAM-1 immunostaining. FIG. 3B: Quantification of the fluorescent signal using Metamorph imaging software *p<0.001.

FIGS. 4A-4D demonstrate that the NLRP3 subunit of the inflammasome and its downstream effector caspase-1 decreased post PIP-2 treatment, in accordance with some embodiments. Cells were pre-treated for 3 hrs with PIP-2 and then with COVID-19 serum/normal serum for an hour. Cells were then fixed and NLRP3/caspase-1 were monitored by immunostaining with anti-NLRP3 or activated caspase-1 antibody. FIG. 4A: NLRP3 immunostaining. FIG. 4B: Quantification of the fluorescent signal using Metamorph imaging software *p<0.001. FIG. 4C: Caspase-1 immunostaining. FIG. 4D: Quantification of the fluorescent signal using Metamorph imaging software *p<0.05.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

COVID-19 has been described largely as a respiratory disease as the respiratory tract and alveoli are amongst the primary sites of infection. One pathology sometimes associated with COVID-19 is the endothelial activation and dysfunction. Endothelial cells are present in the inner layer of blood vessels, and are normally protected by pericytes that support the vessel structure. As the endothelium activates, it becomes the converging site of the inflammation, which involves the expression of adhesion molecules and cytokines and immune cell recruitment. This activation can drive a proinflammatory, procoagulant and prothromobotic phenotype, which can cause damage to lung and other organs. Indeed, pulmonary endothelium activation is often associated with the severity of COVID-19 in patients.

The present study recognizes that agents that can reduce pulmonary endothelial activation and endothelial inflammatory phenotype may be administered during and post COVID-19 infection to maintain endothelial health and function, thereby preventing or treating endothelial activation and dysfunction, as well as reducing the severity of the disease.

The present study hypothesized that the pulmonary endothelium, upon exposure to the “inflammatory load” of the systemic circulation, generates reactive oxygen species (ROS). To test this hypothesis, the present study recreated COVID-19 in vitro by exposing human lung endothelial cells to sera from COVID-19 patients' and control (healthy subjects') sera. The present study discovered that both endothelial activation (as monitored by ROS production) and pro-inflammatory phenotype (as assessed by ICAM-1 and the NLRP3 inflammasome), were significantly higher with COVID-19 as compared to normal subjects.

The present study further discovered that a non-limiting exemplary of peptides that inhibit the NADPH oxidase 2 (NOX2) activation pathway, PIP-2, is able to reduce the endothelial activation.

Accordingly, in some embodiments, the present invention is directed to a method of treating, ameliorating and/or preventing endothelial dysfunction caused by severe acute respiratory syndrome coronavirus 2 (SARS-CoV-2) in a subject in need thereof.

Since endothelial dysfunction, as well as the “inflammatory load” of the systemic circulation, is commonly found in coronavirus infections (e.g., SARS-CoV, MERS-CoV, SARS-CoV-2, etc), it is expected that the method discovered by the present study would be effective in treating and/or ameliorating coronavirus infections.

Accordingly, in some embodiments, the present invention is directed to a method of treating and/or ameliorating COVID-19 in a subject in need thereof.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, “PIP-2” means a peptide having SEQ ID NO: 1 LHDFRHQIL.

As used herein, “PIP-4” means a peptide having SEQ ID NO: 2 LYEIKHQIL.

As used herein, “PIP-5” means a peptide having SEQ ID NO: 3 LYDIRHQIL.

As used herein, “treating a disease or disorder” means reducing the frequency with which a symptom of the disease or disorder is experienced by a patient. Disease and disorder are used interchangeably herein.

As used herein, “sepsis” is a potentially life-threatening condition caused by the body's response to an infection and can lead to multiple organ failure.

As used herein, the term “treatment” or “treating” encompasses prophylaxis and/or therapy. Accordingly, the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Abbreviations: COVID-19: coronavirus disease 2019. HPMVEC: human pulmonary microvascular endothelial cell. ROS: reactive oxygen species. ICAM-1: Intercellular Adhesion Molecule-1. SARS-CoV-2: severe acute respiratory syndrome coronavirus 2.

Method of Treating, Ameliorating, and/or Preventing Endothelial Dysfunction Caused by SARS-CoV2 Infection

In some embodiments, the present invention is directed to a method of treating, ameliorating, and/or preventing endothelial dysfunction caused by severe acute respiratory syndrome coronavirus 2 (SARS-CoV-2) in a subject in need thereof.

In some embodiments, the method includes administering to the subject an effective amount of a polypeptide able to inhibit NADPH oxidase 2 (NOX2) activation pathway. In some embodiments, the polypeptide is able to inhibit a phospholipase A2 (PLA2) activity of peroxiredoxin 6 (Prdx6). In some embodiments, the polypeptide is the same as or similar to those as described in the World Intellectual Property Organization (WIPO) International Publication No. WO 2020/037146 A1. The entirety of this reference is hereby incorporated herein by reference.

In some embodiments, the polypeptide consists of SEQ ID NO: 4

X¹X²X³X⁴X⁵LX⁶X⁷X⁸X⁹HQIL;

wherein:
   X¹ may be present or absent and when present is E;
   X² may be present or absent and when present is L;
   X³ may be present or absent and when present is Q;
   X⁴ may be present or absent and when present is A or T;
   X⁵ is T or E;
   X⁶ is H or Y;
   X⁷ is D or E;
   X⁸ is F or I;
   X⁹ is R or K

As described in WO 2020/037146 A1, the polypeptide having the sequence set forth in SEQ ID NO: 4 is able to inhibit a phospholipase A2 (PLA2) activity of peroxiredoxin 6 (Prdx6). The present study has discovered, using the exemplary Prdx6 inhibitory polypeptide PIP-2 (which has the sequence set forth in SEQ ID NO: 1 LHDFRHQIL), that inhibiting the PLA2 activity of Prdx6 is able to significantly reverse the increased ROS level and the pro-inflammatory phenotype (i.e. intercellular adhesion molecule or ICAM-1) in pulmonary endothelial cells that were induced by COVID-19 sera.

In some embodiments, the polypeptide is selected from the group consisting of:

(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

In some embodiments, the polypeptide is formulated in a liposome.

In some embodiments, the polypeptide is formulated in a pharmaceutical composition comprising a pharmaceutically acceptable carrier.

In some embodiments, administering the polypeptide to the subject comprises aerosol inhalation, intratracheal injection, intravenous injection, or any combinations thereof.

In some embodiments, the endothelial dysfunction comprises endothelial activation and/or endothelial inflammatory phenotype.

In some embodiments, the endothelial dysfunction occurs at least in pulmonary endothelial tissue of the subject.

In some embodiments, the administration results in prevention of reactive oxygen species (ROS) production from cells of the pulmonary endothelial tissue, or reduction of ROS production compared to a reference level of ROS production prior to administering the polypeptide.

In some embodiments, the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

In some embodiments, the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide.

In some embodiments, the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide.

In some embodiments, the administration results in prevention of NLR family pyrin domain containing 3 (NLRP3) inflammasome. In some embodiments, the administration results in reduced expression of the NLRP3 subunit of the inflammasome and/or the reduced expression and/or activation of caspase-1 (downstream of the NLRP3 inflammasome) from cells of the pulmonary endothelial tissue, or reduction of NLRP3 subunit/caspase-1 level compared to a reference level of NLRP3 subunit/caspase-1 level prior to administering the polypeptide.

The NLRP3 inflammasome is an inflammation moiety that comprises the NLRP3 subunit which assembles with its adaptor protein to form a complex that recruits and activates caspase-1. Thus, presence of NLRP3 subunit and activated caspase-1 is indicative of NLRP3 inflammasome activation.

In some embodiments, the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the reduction of caspase-1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the subject is a mammal. In some embodiments, the mammal is selected from the group consisting of a human, a non-human primate, a cow, a pig, a horse, a sheep, a deer, a rabbit, an otter, a mink, a vole, a ferret, a bat, a raccoon dog, a feline, a dog, a hamster, a rat, and a mouse. In some embodiments the subject is a human.

In some embodiments, the subject has active SARS-CoV-2 infection, coronavirus disease 2019 (COVID-19), and/or post-acute sequelae SARS-CoV-2 infection (PASC).

In some embodiments, the SARS-CoV-2 is a SARS-CoV-2 variant.

In some embodiments, the SARS-CoV-2 variant is selected from alpha (B.1.1.7 and Q lineages), beta (B.1.351 and descendent lineages), gamma (P.1 and descendent lineages), delta (B.1.617.2 and AY lineages), epsilon (B.1.427 and B.1.429), zeta (P.2), cta (B.1.525), iota (B.1.526), kappa (B.1.617.1), 1.617.3, mu (B.1.621 and B.1.621.1), and omicron (B.1.1.529 and BA lincages (BA.1, BA.1.1, and BA.2)).

Method of Treating and/or Ameliorating Coronavirus Infection

In some embodiments, the present invention is directed to a method of treating and/or ameliorating coronavirus infection in a subject in need thereof.

In some embodiments, the method includes administering to the subject an effective amount of a polypeptide able to inhibit NADPH oxidase 2 (NOX2) activation pathway. In some embodiments, the polypeptide is able to inhibit a phospholipase A2 (PLA2) activity of peroxiredoxin 6 (Prdx6).

In some embodiments, the polypeptide is the same as or similar to those as described elsewhere herein, such as in the “Method of Treating, Ameliorating, and/or Preventing Endothelial Dysfunction Caused by SARS-CoV2 Infection” section.

In some embodiments, the polypeptide is formulated in a liposome.

In some embodiments, the polypeptide is formulated in a pharmaceutical composition comprising a pharmaceutically acceptable carrier.

In some embodiments, administering the polypeptide to the subject comprises aerosol inhalation, intratracheal injection, intravenous injection, or any combinations thereof.

In some embodiments, the endothelial dysfunction comprises endothelial activation and/or endothelial inflammatory phenotype.

In some embodiments, the coronavirus causes endothelial dysfunction in the subject.

In some embodiments, the endothelial dysfunction occurs at least in pulmonary endothelial tissue of the subject.

In some embodiments, the administration results in prevention of reactive oxygen species (ROS) production from cells of the pulmonary endothelial tissue, or reduction of ROS production compared to a reference level of ROS production prior to administering the polypeptide.

In some embodiments, the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

In some embodiments, the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide.

In some embodiments, the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide.

In some embodiments, the administration results in prevention of NLR family pyrin domain containing 3 (NLRP3) inflammasome. In some embodiments, the administration results in reduced expression of the NLRP3 subunit of the inflammasome and/or the reduced expression and/or activation of caspase-1 (downstream of the NLRP3 inflammasome) from cells of the pulmonary endothelial tissue, or reduction of NLRP3 subunit/caspase-1 level compared to a reference level of NLRP3 subunit/caspase-1 level prior to administering the polypeptide.

In some embodiments, the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the reduction of caspase-1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide.

In some embodiments, the subject is a mammal. In some embodiments, the mammal is selected from the group consisting of a human, a non-human primate, a cow, a pig, a horse, a sheep, a deer, a rabbit, an otter, a mink, a vole, a ferret, a bat, a raccoon dog, a feline, a dog, a hamster, a rat, and a mouse. In some embodiments the subject is a human.

In some embodiments, the coronavirus is at least one selected from the group consisting of SARS-CoV, MERS-CoV, SARS-CoV2, HCoV-OC43, HCoV-HKU1, HCoV-229E, and HCoV-NL63.

### Combination Therapies

The compounds useful within the methods described herein can be used in combination with one or more additional therapeutic agents useful for treating a coronavirus infection, such as a SARS-CoV2 infection. These additional therapeutic agents may comprise compounds that are commercially available or synthetically accessible to those skilled in the art. These additional therapeutic agents are known to treat or reduce the symptoms, of a coronavirus infection, such as a SARS-CoV2 infection.

In certain embodiments, the compounds described herein can be used in combination with radiation therapy. In other embodiments, the combination of administration of the compounds described herein and application of radiation therapy is more effective in treating or preventing a coronavirus infection, such as a SARS-CoV2 infection than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a coronavirus infection, such as a SARS-CoV2 infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a coronavirus infection, such as a SARS-CoV2 infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat a coronavirus infection, such as a SARS-CoV2 infection in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for case of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a coronavirus infection, such as a SARS-CoV2 infection, in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a coronavirus infection, such as a SARS-CoV2 infection in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

It is believed that the endothelium that lines the vascular network is affected during and post COVID-19. This effect is considered “indirect” and arises from the inflammation and oxidative stress load in the systemic circulation. Endothelial activation for long periods leads to endothelial dysfunction and if uncontrolled will drive vessel leakage, edema, cell death and finally organ damage.

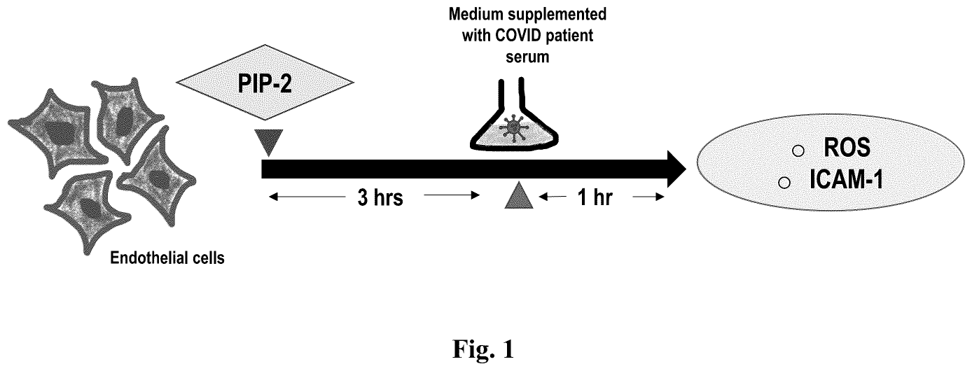

Referring to FIG. 1, the study described herein (“the present study”) developed a pre-clinical COVID-19 model using serum collected from patients that tested SARS-CoV2 positive at ICU admission. The present study then tested a novel peptide PIP-2 for the ability to protected the endothelium from activation when the PIP-2 is administered pre-COVID-19 serum application. The results showed that pre-treatment (prophylactic paradigm) protected the endothelium from activation (ROS production) and prevented the appearance of an endothelial inflammatory phenotype (ICAM-1 expression). It is expected that the PIP-2 treatment at the same time at or after COVID-19 serum applications would result in significant decrease in endothelial activation (as monitored by ROS production and endothelial inflammatory phenotype), as well. This is because COVID-19 serum appears to initiate an endothelial activation pathway, the sequelae to which is the appearance of an endothelial inflammatory phenotype. PIP-2's intervention at any point after initiation will impact the activation and halt the progression of this pathway.

### Example 2: Overview

Recent research suggests that endothelial activation plays a role in COVID-19 pathogenesis by promoting a pro-coagulative and pro-inflammatory state. However, the mechanism by which the endothelium is activated in COVID-19 is unclear. Thus, one of the goals of the instant study is to investigate the mechanism by which COVID-19 activates the pulmonary endothelium so as to be able to abrogate it.

It was hypothesized that the pulmonary endothelium, upon exposure to the “inflammatory load” of the systemic circulation, generates reactive oxygen species (ROS) via a NADPH oxidase 2 (NOX2) activation pathway. To test this hypothesis, COVID-19 was recreated in vitro and ex vivo, by exposing human lung endothelial cells and lung slices (human precision-cut lung slices or huPCLS) to serum from COVID-19 affected subjects (sera were acquired from patients with COVID-19 infection admitted to the Intensive Care Unit of the Hospital at the University of Pennsylvania). This was followed by assessment of ROS (fluorescent dye, CellROX) and intercellular adhesion molecule (ICAM-1) by fluorescence labeling and imaging.

The present study discovered that both endothelial activation (as monitored by ROS production) and pro-inflammatory phenotype (as assessed by ICAM-1), were significantly higher with COVID-19 as compared to normal subjects. Pre-treatment of cells with novel peptide that blocks the PLA2 activity of NOX2 significantly reduced ROS and ICAM-1. These results support that the endothelium is activated with COVID-19 via NOX2 initiated ROS production; the ROS thus produced drives a pro-inflammatory phenotype by inducing the expression of ICAM-1, a pivotal marker of endothelium inflammation. As ROS mediates endothelial activation and inflammation during COVID-19, usage of PIP-2 could have therapeutic implications in maintaining vascular health.

### Example 3: Related Information

The vascular endothelium is an interface between blood and tissues and thus by virtue of its location exposed to the inflammatory signals in blood. On encountering either bloodborne pathogen or inflammatory moieties, the endothelial cells trigger the host defenses in the form of upregulation of adhesion molecules and chemokine and cytokine production thus sending warning signs of infection, invasion or injury. While this enables the endothelium is to regulate and co-ordinate the host defense system, this also leads to a diseased state as endothelial homeostasis is disrupted. The normal endothelium possesses anti-inflammatory, anticoagulant, antithrombotic, and profibrinolytic properties that maintain homeostasis; alteration in these promote inflammation, adhesive properties in the endothelium and thrombus accumulation.

COVID-19 has been described largely as a respiratory disease; indeed, the respiratory tract and alveoli are amongst the primary sites of infection. However, it is also an inflammatory disease where release of inflammatory cytokines is the cause of organ injury and damage. The endothelium is the converging site of the inflammation as its activation (expression of adhesion molecules and cytokines) leads to immune cell recruitment; thus, it is reasonable to conclude that COVID-19 is potentially a vascular disease that has its origins in “endothelial inflammation” signaling.

Inspection of lungs of fatal cases of COVID-19 revealed the appearance of inflammation along the vascular endothelial wall as well as the presence of extensive microthrombi throughout the lung. The SARS-CoV2 virus does not seem to cross the epithelial barrier and reach the systemic circulation, indicating that there are possibly no direct effects of the infection on endothelium (see e.g., Paul et al, Respir Res. 2022 Feb. 10; 23 (1): 25; Schimmel, Clin Transl Immunology. 2021 Oct. 24; 10 (10): e1350). Indirect effects on the endothelium stem from the cytokine storm associated with the immune response which cause a high “inflammation load” on the circulatory system. It was hypothesized that exposure of the endothelial network to this “inflammation lead” can activate endothelial cells. This activation can drive a proinflammatory, procoagulant and prothromobotic phenotype. Therefore, agents that can reduce endothelial activation and endothelial inflammatory phenotype may be administered during and post COVID-19 infection to maintain endothelial health and function.

The major source of ROS in the lung endothelium is NADPH oxidase type 2 (NOX2) that is expressed especially in endothelial cells, polymorphonuclear leucocytes (PMN) and alveolar macrophages, but also in lung epithelium. This normally quiescent enzyme requires activation via a complex pathway that includes the binding of cytoplasmic factors p40, p47, p67, and Rac in order to generate ROS. The activation of NOX2 in endothelium requires the phospholipase A2 activity of peroxiredoxin 6 (Prdx6) and that inhibition of this activity (called aiPLA₂ activity) largely prevents ROS generation by endothelial lung cells. To inhibit aiPLA₂ activity pharmacologically, a non-specific PLA₂ inhibitor, called MJ33 was initially used and found to be able to protect against lung oxidative injury. A 9 aa peptide within the 16 aa sequence as the minimal sequence for inhibition of aiPLA₂ activity was then developed. This peptide was named “peroxiredoxin 6-inhibitory peptide-2” or “PIP-2,” and appears to be specific for inhibition of aiPLA₂ activity.

The present study used the novel peptide PIP-2 on a pre-clinical model of COVID-19, which recreated the vascular environment of COVID-19 affected subjects by treating human pulmonary endothelial cells with serum from COVID-19 patients. The present study assessed endothelial activation by monitoring the production of reactive oxygen species (ROS). The present study also ascertained the appearance of an endothelial inflammatory phenotype by assessment of intercellular adhesion molecule (ICAM-1).

### Example 4: Materials and Methods

### Cell Culture

Primary cultures of Human Pulmonary Microvascular Endothelial Cells (HPMVEC) were grown in a T-25 culture dish and routinely maintained in Dulbecco's Modified Eagle Medium (DMEM) supplemented with 10% fetal bovine serum (FBS), 1× glutamax, 1× antibiotics, and endothelial cell growth supplement (Millipore Upstate). Cells were grown and maintained in 5% CO₂ at 37° C. Once grown to the appropriate density, cells were then split and grown on coverslips in 35 mm petri dishes. Cells were then treated either with medium supplemented with 10% serum from healthy individuals or COVID-19 patients at admission to ICU and then monitored for ROS production and ICAM-1 induction as described below.

### Serum Treatment

HPMVEC were incubated for one hour in the incubator at 37° C. and 5% CO₂ with medium supplemented with 10% serum either from a healthy individual or from patients suffering from COVID-19. The serum replaced the FBS which was otherwise used as a supplement in DMEM. Post incubation, cells were either fixed for ICAM-1 immuno-staining or the cells were stained for ROS measurement as described below. Serum from one healthy individual and seven COVID-19 patients were used in this study.

### PIP-2 Treatment

HPMVEC were incubated for three hours in the incubator at 37° C. and 5% CO₂ with medium supplemented with either liposomes alone or PIP-2 in liposomes (concentration 10 nM) without 10% FBS. After 3 hours of incubation, cells were either treated with serum of a healthy individual or COVID-19 patients at admission to ICU along with either liposomes alone or PIP-2 in liposomes together for one hour and three hours for ROS measurement and ICAM-1 induction respectively.

### ROS Measurement

After the 1-hour incubation, the medium was replaced with fresh medium without FBS and the serum treated cells were labeled using CellROX green, 10 μM for one hour at 37° C. and 5% CO₂. CellROX is a ROS sensitive dye. Post one hour staining, cells were imaged for fluorescence using Nikon TMD epifluorescence microscope equipped with a Hamamatsu ORCA-100 digital camera and Metamorph imaging software (Universal Imaging, West Chester, PA). All images were taken using 20× lens and at Aex=488 nm. The same procedure was followed for ROS measurement in PIP-2 serum treated cells.

### ICAM-1 Immunostaining

For the measurement of ICAM-1 induction, the cells were treated with medium supplemented with serum for 3 hrs at 37° C. and 5% CO₂. Post 3 hrs incubation, serum treated cells were washed with 1× phosphate buffered saline (PBS) and were then fixed with 4% paraformaldehyde for 10 minutes, followed by permeabilization. The permeabilized HPMVEC were immunostained with anti-ICAM (1:150; ab 171123, abcam) overnight for two days. Anti-mouse IgG conjugated to Alexa 488 (green) was used as the secondary antibody (1:200) for 1 hr. Vectashield antifade vibrance mounting medium was used to mount the cells on glass coverslips. Imaging was done using Nikon TMD epifluorescence microscope equipped with a Hamamatsu ORCA-100 digital camera and Metamorph imaging software (Universal Imaging, West Chester, PA). All images were taken using 20× lens and at Aex=488 nm. All images were acquired with the same exposure and acquisition settings. The same procedure was followed for ICAM-1 measurement in PIP-2 serum treated cells.

### Imaging for PIP-2 Treatment Cells

Leica STED super resolution laser microscope with excitation and emission filters for was λex=488 nm and λem=500-560 nm was used for imaging serum treated cells with either liposome alone or with PIP-2 in liposomes. LASX software was used for acquiring all the images under the same setting.

### Example 5: Results

The present study evaluated the reactive oxygen species (ROS) production in the endothelial cells upon COVID-19 serum exposure.

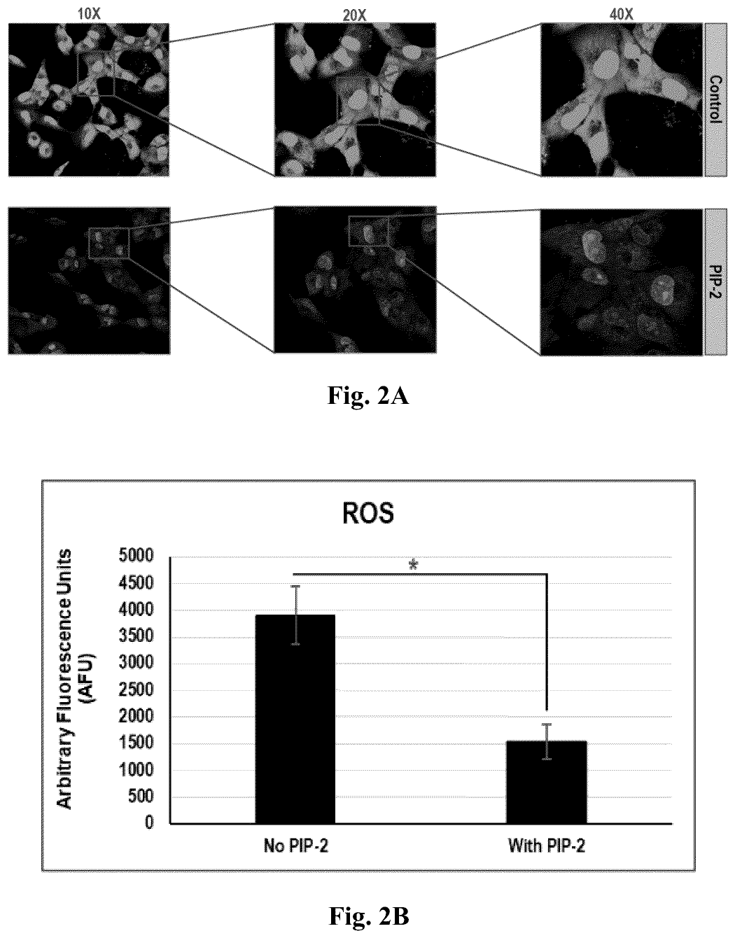

Referring to FIGS. 2A-2B, pre-treatment of the endothelial cells with PIP-2 led to a significant decrease in ROS production post COVID-19 serum exposure.

Next, the present study evaluated expression of ICAM-1, an adhesion molecule that facilitates recruitment and adherence of immune cells to the endothelium. ICAM-1 is often considered as a surrogate of inflammation.

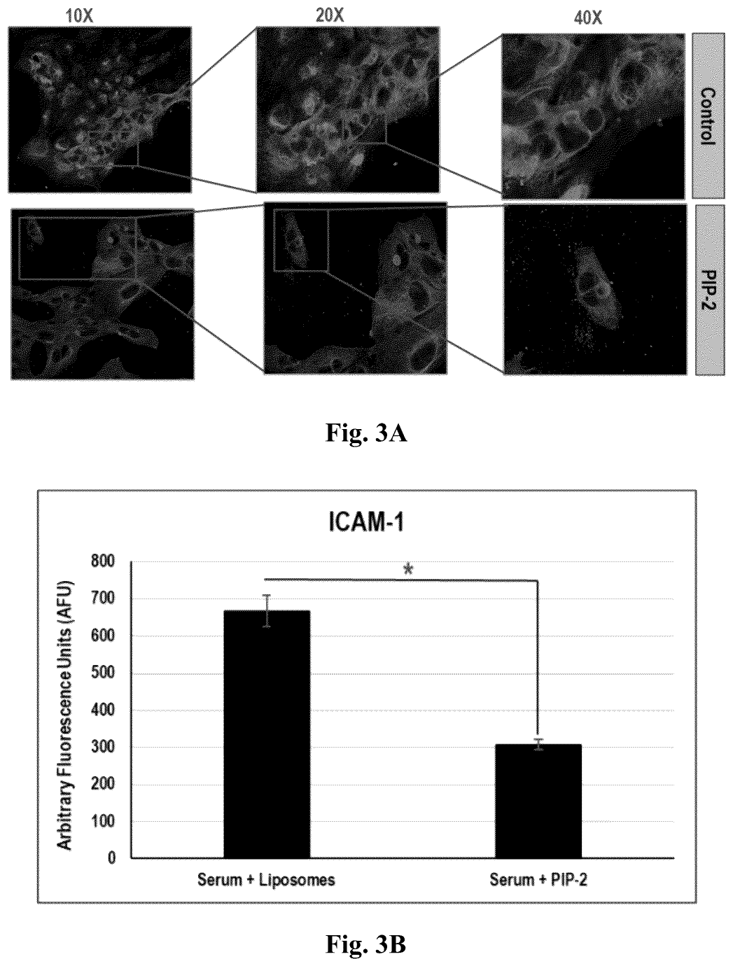

Referring to FIGS. 3A-3B, ICAM-1 expression was significantly reduced in samples which were pre-treated with PIP-2.

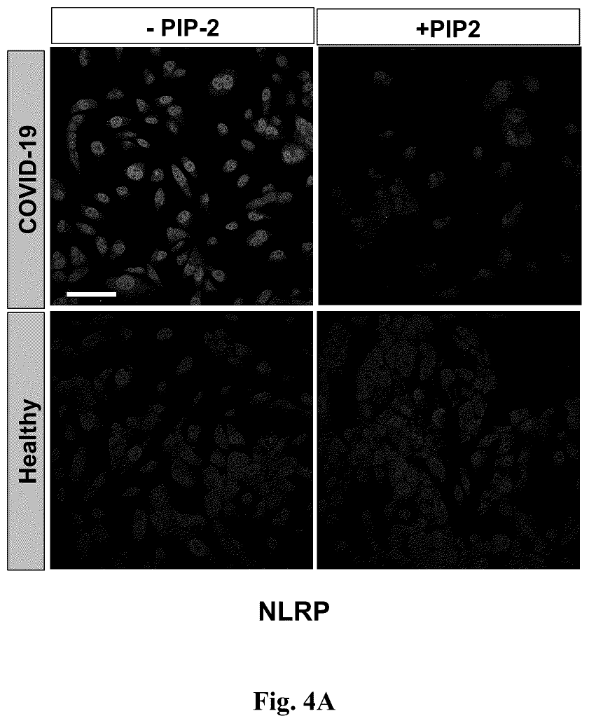

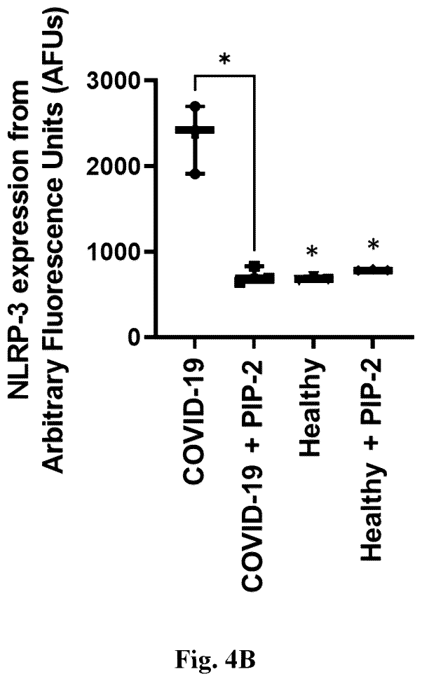

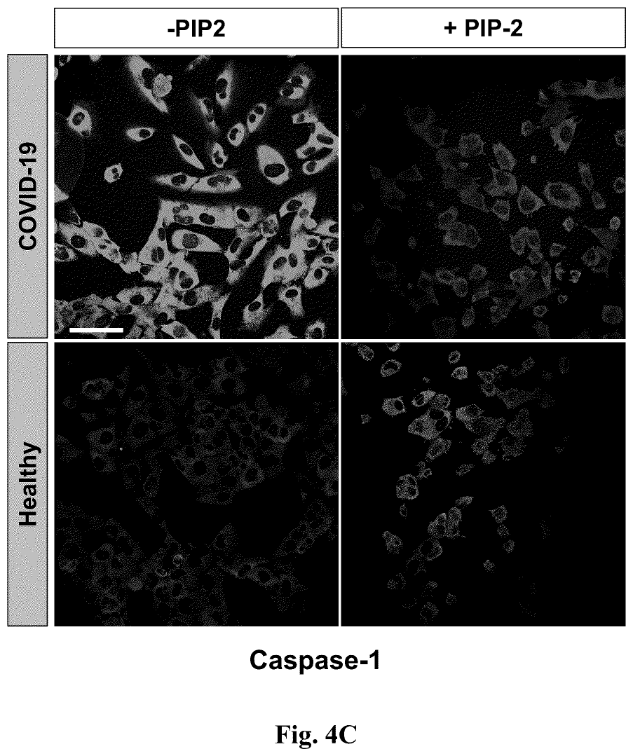

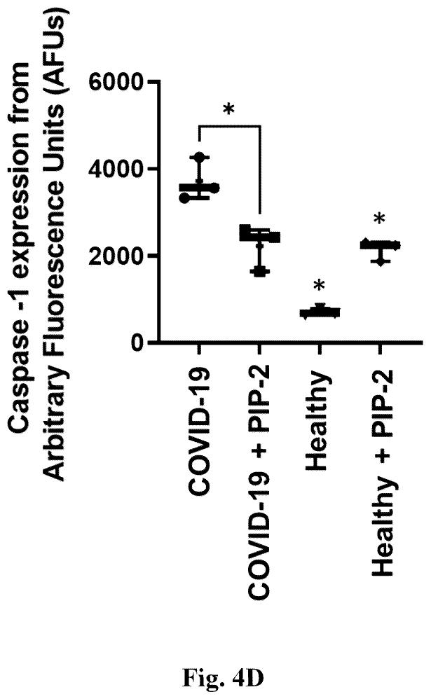

Referring to FIGS. 4A-4B, NLRP3 (a component of the inflammasome which mediates the activation of caspase-1) expression was significantly reduced in samples which were pre-treated with PIP-2.

Referring to FIGS. 4C-4D, caspase 1 expression was significantly reduced in samples which were pre-treated with PIP-2.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

### ENUMERATED EMBODIMENTS

In some aspects, the present invention is directed to the following non-limiting embodiments:

Embodiment 1: A method of treating, ameliorating, and/or preventing endothelial dysfunction caused by severe acute respiratory syndrome coronavirus 2 (SARS-CoV-2) in a subject in need thereof, the method comprising administering to the subject an effective amount of a polypeptide consisting of SEQ ID NO: 4

X¹X²X³X⁴X⁵LX⁶X⁷X⁸X⁹HQIL;

wherein:
   X¹ may be present or absent and when present is E;
   X² may be present or absent and when present is L;
   X³ may be present or absent and when present is Q;
   X⁴ may be present or absent and when present is A or T;
   X⁵ is T or E;
   X⁶ is H or Y;
   X⁷ is D or E;
   X⁸ is F or I;
   X⁹ is R or K.

Embodiment 2: The method of Embodiment 1, wherein the polypeptide is selected from the group consisting of:

(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

Embodiment 3: The method of Embodiment 1 or Embodiment 2, wherein the polypeptide is formulated in a liposome.

Embodiment 4: The method of any one of Embodiments 1-3, wherein the polypeptide is formulated in a pharmaceutical composition comprising a pharmaceutically acceptable carrier.

Embodiment 5: The method of any one of Embodiments 1-4, wherein administering the polypeptide to the subject comprises aerosol inhalation, intratracheal injection, intravenous injection, or any combinations thereof.

Embodiment 6: The method of any one of Embodiments 1-5, wherein the endothelial dysfunction comprises endothelial activation and/or endothelial inflammatory phenotype.

Embodiment 7: The method of any one of Embodiments 1-6, wherein the endothelial dysfunction occurs at least in pulmonary endothelial tissue of the subject.

Embodiment 8: The method of Embodiment 7, wherein the administration results in prevention of reactive oxygen species (ROS) production from cells of the pulmonary endothelial tissue, or reduction of ROS production compared to a reference level of ROS production prior to administering the polypeptide.

Embodiment 9: The method of Embodiment 8, wherein the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

Embodiment 10: The method of Embodiment 7, wherein at least one of the following applies:
(a) the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide,
(b) the administration results in prevention of NLR family pyrin domain containing (NLRP3) expression from cells of the pulmonary endothelial tissue, or reduction of NLRP3 expression compared to a reference level of NLRP3 expression prior to administering the polypeptide, or
(c) the administration results in prevention of caspase 1 expression from cells of the pulmonary endothelial tissue, or reduction of caspase 1 expression compared to a reference level of caspase 1 expression prior to administering the polypeptide.

Embodiment 11: The method of Embodiment 10, wherein at least one of the following applies:
(a) the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide,
(b) the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide, or
(c) the reduction of caspase 1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 expression prior to administering the polypeptide.

Embodiment 12: The method of any one of Embodiments 1-11, wherein the subject is a mammal.

Embodiment 13: The method of Embodiment 12, wherein the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

Embodiment 14: The method of Embodiment 13, wherein the subject is a human.

Embodiment 15: The method of any one of Embodiments 1-14, wherein the subject has active SARS-CoV-2 infection, coronavirus disease 2019 (COVID-19), and/or post-acute sequelae SARS-CoV-2 infection (PASC).

Embodiment 16: The method of any one of Embodiments 1-15, wherein the SARS-CoV-2 is a SARS-CoV-2 variant.

Embodiment 17: The method of Embodiment 16, wherein the SARS-CoV-2 variant is selected from alpha (B.1.1.7 and Q lineages), beta (B.1.351 and descendent lineages), gamma (P.1 and descendent lineages), delta (B.1.617.2 and AY lineages), epsilon (B.1.427 and B.1.429), zeta (P.2), eta (B.1.525), iota (B.1.526), kappa (B.1.617.1), 1.617.3, mu (B.1.621 and B.1.621.1), and omicron (B.1.1.529 and BA lineages (BA.1, BA.1.1, and BA.2)).

Embodiment 18: A method of treating or ameliorating a coronavirus infection in a subject in need thereof, the method comprising administering to the subject an effective amount of a polypeptide consisting of SEQ ID NO: 4

X¹X²X³X⁴X⁵LX⁶X⁷X⁸X⁹HQIL;

wherein:
   X¹ may be present or absent and when present is E;
   X² may be present or absent and when present is L;
   X³ may be present or absent and when present is Q;
   X⁴ may be present or absent and when present is A or T;
   X⁵ is T or E;
   X⁶ is H or Y;
   X⁷ is D or E;
   X⁸ is F or I;
   X⁹ is R or K.

Embodiment 19: The method of Embodiment 18, wherein the polypeptide is selected from the group consisting of:

(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

Embodiment 20: The method of Embodiment 18 or Embodiment 19, wherein the polypeptide is formulated in a liposome.

Embodiment 21: The method of any one of Embodiments 18-20, wherein the polypeptide is formulated in a pharmaceutical composition comprising a pharmaceutically acceptable carrier.

Embodiment 22: The method of any one of Embodiments 18-21, wherein administering the polypeptide to the subject comprises aerosol inhalation, intratracheal injection, intravenous injection, or any combinations thereof.

Embodiment 23: The method of any one of Embodiments 18-22, wherein the coronavirus infection causes endothelial dysfunction in the subject.

Embodiment 24: The method of Embodiment 23, wherein the endothelial dysfunction comprises endothelial activation and/or endothelial inflammatory phenotype.

Embodiment 25: The method of any one of Embodiments 23-24, wherein the endothelial dysfunction occurs at least in pulmonary endothelial tissue of the subject.

Embodiment 26: The method of Embodiment 25, wherein the administration results in prevention of reactive oxygen species (ROS) production from cells of the pulmonary endothelial tissue, or reduction of ROS production compared to a reference level of ROS production prior to administering the polypeptide.

Embodiment 27: The method of Embodiment 26, wherein the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

Embodiment 28: The method of Embodiment 25, wherein at least one of the following applies:
(a) the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide,
(b) the administration results in prevention of NLR family pyrin domain containing (NLRP3) expression from cells of the pulmonary endothelial tissue, or reduction of NLRP3 expression compared to a reference level of NLRP3 expression prior to administering the polypeptide, or
(c) the administration results in prevention of caspase 1 expression from cells of the pulmonary endothelial tissue, or reduction of caspase 1 expression compared to a reference level of caspase 1 expression prior to administering the polypeptide.

Embodiment 29: The method of Embodiment 28, wherein at least one of the following applies:
(a) the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide,
(b) the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide, or
(c) the reduction of caspase 1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 expression prior to administering the polypeptide.

Embodiment 30: The method of any one of Embodiments 18-29, wherein the subject is a mammal.

Embodiment 31: The method of Embodiment 30, wherein the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

Embodiment 32: The method of Embodiment 31, wherein the subject is a human.

Embodiment 33: The method of any one of Embodiments 18-32, wherein the coronavirus comprises at least one selected from the group consisting of SARS-CoV, MERS-CoV, SARS-CoV2, HCoV-OC43, HCoV-HKU1, HCOV-229E, and HCoV-NL63.

What is claimed is:

1. A method of treating, ameliorating, and/or preventing endothelial dysfunction caused by severe acute respiratory syndrome coronavirus 2 (SARS-CoV-2) in a subject in need thereof, the method comprising administering to the subject an effective amount of a polypeptide consisting of SEQ ID NO: 4
X¹X²X³X⁴X⁵LX⁶X⁷X⁸X⁹HQIL;
wherein:
X¹ may be present or absent and when present is E;
X² may be present or absent and when present is L;
X³ may be present or absent and when present is Q;
X⁴ may be present or absent and when present is A or T;
X⁵ is T or E;
X⁶ is H or Y;
X⁷ is D or E;
X⁸ is F or I;
X⁹ is R or K.

2. The method of claim 1, wherein the polypeptide is selected from the group consisting of:
(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

3. The method of claim 1, wherein the polypeptide is formulated in a liposome.

4. The method of claim 1, wherein the polypeptide is formulated in a pharmaceutical composition comprising a pharmaceutically acceptable carrier.

5. The method of claim 1, wherein administering the polypeptide to the subject comprises aerosol inhalation, intratracheal injection, intravenous injection, or any combinations thereof.

6. The method of claim 1, wherein the endothelial dysfunction comprises endothelial activation and/or endothelial inflammatory phenotype.

7. The method of claim 1, wherein the endothelial dysfunction occurs at least in pulmonary endothelial tissue of the subject.

8. The method of claim 7, wherein the administration results in prevention of reactive oxygen species (ROS) production from cells of the pulmonary endothelial tissue, or reduction of ROS production compared to a reference level of ROS production prior to administering the polypeptide.

9. The method of claim 8, wherein the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

10. The method of claim 7, wherein at least one of the following applies:
(a) the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide,
(b) the administration results in prevention of NLR family pyrin domain containing (NLRP3) expression from cells of the pulmonary endothelial tissue, or reduction of NLRP3 expression compared to a reference level of NLRP3 expression prior to administering the polypeptide, or
(c) the administration results in prevention of caspase 1 level from cells of the pulmonary endothelial tissue, or reduction of caspase 1 level compared to a reference level of caspase 1 expression prior to administering the polypeptide.

11. The method of claim 10, wherein at least one of the following applies:
(a) the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide,
(b) the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide, or
(c) the reduction of caspase 1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 level prior to administering the polypeptide.

12. The method of claim 1, wherein the subject is a mammal.

13. The method of claim 12, wherein the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

14. The method of claim 13, wherein the subject is a human.

15. The method of claim 1, wherein the subject has active SARS-CoV-2 infection, coronavirus disease 2019 (COVID-19), and/or post-acute sequelae SARS-CoV-2 infection (PASC).

16. The method of claim 1, wherein the SARS-CoV-2 is a SARS-CoV-2 variant.

17. The method of claim 16, wherein the SARS-CoV-2 variant is selected from alpha (B.1.1.7 and Q lineages), beta (B.1.351 and descendent lineages), gamma (P.1 and descendent lineages), delta (B.1.617.2 and AY lineages), epsilon (B.1.427 and B.1.429), zeta (P.2), eta (B.1.525), iota (B.1.526), kappa (B.1.617.1), 1.617.3, mu (B.1.621 and B.1.621.1), and omicron (B.1.1.529 and BA lineages (BA.1, BA.1.1, and BA.2)).

18. A method of treating or ameliorating a coronavirus infection in a subject in need thereof, the method comprising administering to the subject an effective amount of a polypeptide consisting of SEQ ID NO: 4
X¹X²X³X⁴X⁵LX⁶X⁷X⁸X⁹HQIL;
wherein:
X¹ may be present or absent and when present is E;
X² may be present or absent and when present is L;
X³ may be present or absent and when present is Q;
X⁴ may be present or absent and when present is A or T;
X⁵ is T or E;
X⁶ is H or Y;
X⁷ is D or E;
X⁸ is F or I;
X⁹ is R or K.

19. The method of claim 18, wherein the polypeptide is selected from the group consisting of:
(i)
SEQ ID NO: 1
LHDFRHQIL;
(ii)
SEQ ID NO: 2
LYEIKHQIL;
(iii)
SEQ ID NO: 3
LYDIRHQIL;
(iv)
SEQ ID NO: 5
ELQTELYEIKHQIL;
(v)
SEQ ID NO: 6
QTELYEIKHQIL;
and
(vi)
SEQ ID NO: 7
ELYEIKHQIL.

20. The method of claim 18, wherein the polypeptide is formulated in a liposome.

21. The method of claim 18, wherein the polypeptide is formulated in a pharmaceutical composition comprising a pharmaceutically acceptable carrier.

22. The method of claim 18, wherein administering the polypeptide to the subject comprises aerosol inhalation, intratracheal injection, intravenous injection, or any combinations thereof.

23. The method of claim 18, wherein the coronavirus infection causes endothelial dysfunction in the subject.

24. The method of claim 23, wherein the endothelial dysfunction comprises endothelial activation and/or endothelial inflammatory phenotype.

25. The method of claim 23, wherein the endothelial dysfunction occurs at least in pulmonary endothelial tissue of the subject.

26. The method of claim 25, wherein the administration results in prevention of reactive oxygen species (ROS) production from cells of the pulmonary endothelial tissue, or reduction of ROS production compared to a reference level of ROS production prior to administering the polypeptide.

27. The method of claim 26, wherein the reduction of ROS production is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ROS production prior to administering the polypeptide.

28. The method of claim 25, wherein at least one of the following applies:
(a) the administration results in prevention of Intercellular Adhesion Molecule 1 (ICAM-1) expression from cells of the pulmonary endothelial tissue, or reduction of ICAM-1 expression compared to a reference level of ICAM-1 expression prior to administering the polypeptide,
(b) the administration results in prevention of NLR family pyrin domain containing (NLRP3) expression from cells of the pulmonary endothelial tissue, or reduction of NLRP3 expression compared to a reference level of NLRP3 expression prior to administering the polypeptide, or
(c) the administration results in prevention of caspase 1 activation from cells of the pulmonary endothelial tissue, or reduction of caspase 1 level compared to a reference level of caspase 1 level prior to administering the polypeptide.

29. The method of claim 28, wherein at least one of the following applies:
(a) the reduction of ICAM-1 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of ICAM-1 expression prior to administering the polypeptide,
(b) the reduction of NLRP3 expression is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of NLRP3 expression prior to administering the polypeptide, or
(c) the reduction of caspase 1 level is at least a 10% reduction, at least a 20% reduction, at least a 30% reduction, at least a 40% reduction, at least a 50% reduction, at least a 60% reduction, at least a 70% reduction, at least an 80% reduction, at least a 90% reduction, at least a 100% reduction, or more, compared to the reference level of caspase 1 level prior to administering the polypeptide.

30. The method of claim 18, wherein the subject is a mammal.

31. The method of claim 30, wherein the mammal is selected from a human, a non-human primate, a cow, a pig, a horse, a sheep, a deer, a rabbit, an otter, a mink, a vole, a ferret, a bat, a raccoon dog, a feline, a dog, a hamster, a rat, and a mouse.

32. The method of claim 31, wherein the subject is a human.

33. The method of claim 32, wherein the coronavirus comprises at least one selected from the group consisting of SARS-CoV, MERS-CoV, SARS-CoV2, HCoV-OC43, HCoV-HKU1, HCoV-229E, and HCoV-NL63.
